(19) United States
(12) Patent Application Publication
(10) Pub. No.: US 2006/0159633 A1
(43) Pub. Date: Jul. 20, 2006

(54) emulsive water-soluble concentrates

(76) Inventor: Rudi Wajda, Mannheim (DE)

Correspondence Address: STAAS & HALSEY LLP, SUITE 700, 1201 NEW YORK AVENUE, N.W., WASHINGTON, DC, 20005, US

(21) Appl. No.: 10/536,384

(22) PCT Filed: Nov. 25, 2003
(86) PCT No.: PCT/DE03/03887

§ 371 Date: Mar. 14, 2006

(30) Foreign Application Priority Data
Nov. 27, 2002 (DE) 102 55 195.2

(51) Int. Cl. A61K 8/73

(52) U.S. Cl. 424/59

(57) ABSTRACT

The invention relates to the solubilization of lipophilic substances, preferably from the group of lipids, steroids, terpenes and polar lipids by means of a lecithin/polyol matrix or a lecithin/carbohydrate matrix so as to obtain water-soluble emulsion-like transparent concentrates which are used in cosmetic, dietetic and pharmaceutical products.

The present invention relates to emulsion-like transparent to translucent water-soluble concentrates according to the features of claim 1 and their use in cosmetic, pharmaceutical or dietetic products.

Lipophilic or fatlike substances in the pharmaceutical, cosmetic or dietetic field usually have to be transfered into a problem-free application form. An actually problem-free application form is the emulsion with emulsifiers reducing the surface tension at the interface of fat droplets thus providing a fine and stable distribution of fat in water. Depending on the field of application there are emulsions from creamlike to milky consistency with amounts of emulsifiers from 0.5-10% (w/w). The particle size of the fat droplets of a conventional emulsion depends on many factors, like fat, emulsifier, applied energy und is usually within a three digit nm-range (100-1,000 nm).

Special questions and applications demand for transparent to translucent products with sizes of the droplets within a one to two digit nm range (5-100 nm). Such formulations can only be prepared by micellar solubilization. Lipophilic materials are solubilized in form of mixed micells with a mixture of a suitable solubilizer (emulsifier) and a coemulsifier to give transparent formulations.

In the field of cosmetics and dermatology visually esthetical products are needed containing poorly water-soluble substances in partly high concentrations. Besides the appearance such products are supposed to have an excellent physical stability. Examples are transparent bath oils in cosmetics and dermatology with triglycerides, mineral oils and essential oils as fatlike substances.

Perfumes predominantly contain fatlike fragrances as well dissolved in a clear transparent form. Poorly water-soluble pharmaceuticals are also processed to transparent formulations for oral or parenteral applications.

It is the objective of a topical, oral or parenteral applied product to use the lipophilic active substance. Emulsifiers or coemulsifiers are unwanted but to date technologically necessary auxiliary substances.

It is state of the art that the solubilization of fatlike substances to transparent systems in the above mentioned fields of application can be made exclusively with ethoxylated tensides or tensides having a high HLB-value and/or with alcohols. However, these solubilizers have serious deficiencies:
the highly volatile alcohols-VOC-(Ethanol, Isopropanol) are strong cytotoxins and
concerning the protection of the atmosphere they are unwanted in many formulations.
Emulsifiers have to be used in big quantities for the solubilization process so that the application of the actually active substance requires considerable quantities of auxiliary substances. In order to solubilize for example 1 g lavender oil in the common way 5 g PEG 40 hydrogenated castor oil in a 25% (w/w) ethanolic solution are needed. Or in order to solubilize for example 20 g vitamin E-acetate in the common way 44 g PEG 36 hydrogenated castor oil and 25 g propylene glycol in an aqueous solution are needed. And in order to solubilize the lipophilic vitamins A, E and D in the common way a ten times excess of a mixture of glycocholic acid and phosphatidylcholine is needed for example.

DE-198 59 427 A1 discloses the production of micellar dissolved fatlike substances to transparent systems in the form of microemulsions. As a system of emulsifier—/coemulsifier exclusively a mixture of lecithin and ethoxylated emulsifiers, lecithin/emulsifiers with high HLB-value or lecithin/highly volatile alcohols are used. DE 199 22 193 describes the production of a typical milky fat emulsion of hydrogenated lecithin, essential oils and water and the production of optically transparent concentrates.

The present invention has the objective to dissolve poorly water-soluble substances to transparent or translucent emulsion-like water-soluble concentrates and to apply these concentrates.

This objective is achieved with emulsion-like water-soluble concentrates according to the invention with the features of claim 1 and the application of these emulsion-like water-soluble concentrates with the features of claim 10.

Contrary to micellar systems up to date the advantage is achieved with the invention that it refrains completely from a second emulsifier as well as from mono-or dihydric alcohols. Further surprisingly, related to the lipophilic substance, this could be done with a lack of phospholipids. Thus being in contradiction to the conventional possibilities up to date to achieve solubilization.

According to the invention ethoxylated tensides or other strong solubilizers can be replaced by a system of natural substances (lecithins/phospholipids/polyols) being uncritical for the health and safe for the environment.

According to the invention only 5-100% (w/w)of the mass of a fat is needed as solubilizer for the solubilization process (lecithins/phospholipids). With procedures up to date this proportion is inverse.

According to the invention highly volatile organic solubilizers (ethanol, isopropanol, etc.) can be avoided. According to the invention emulsion-like concentrates are created by means of a one step production process resulting in an opaque to transparent, finely dispersed emulsion after dilution with water. This type of emulsion would not or only much more difficult be manufacturable if the emulsion-like concentrate is avoided.

These new concentrates can be produced best by means of a high pressure homogenizer.

Rotor-stator mixers achieve less transparent concentrates. The disadvantage from DE 198 59 427, caused by metal abrasion in high pressure homogenizers, is disposed by using ceramic for homogenisation in respective devices. The concentrates according to the invention can be used directly as products for e.g. medical or cosmetic bath oils, mouthwash, perfumeoils, beverages or food-supplements or can be transferred by dilution with water or other aqueous solutions (e.g. juices) to finely dispersed, transparent—opaque o/W emulsions (nanoemulsions) with very small distribution of particle sizes in the two to three digit nm range. Due to the very good solubilization in water these transparent emulsion-like concentrates can be incorporated without problems into cosmetic products (gel, cream, lotions, etc.) pharmaceutical or dietetic products.

The production of an emulsion-like water-soluble concentrate of lecithins and/or phospholipids, lipids and highly concentrated solutions of polyols or carbohydrates takes place as follows:
The ratio of lecithin/phospholipid to fatlike substance should be chosen in such a way that a transparent concentrate results allowing dilution in water without problems and
the part of water of the polyol—or carbohydrate solution should be chosen in such a way that not only a transparent concentrate results but also—due to the low value of water—a self preserving system of a kind that the addition of synthetic or as well natural preservatives can be avoided.
The process temperature should be adapted to the used lecithins/phospholipids or to the solubilized lipids. Hydrogenated lecithins/phospholipids need naturally process temperatures from 40 to 80° C., unsaturated lecithins/phospholipids can be processed at room temperature the lecithins/phospholipids being integrated preferably into the polar phase, i. e. into the polyol—or carbohydrat solutions.

The present invention is presented by means of preferred examples.

### EXAMPLE 1

5 g of a fraction of phospholipids from soya with a PC-content of 70% is dispersed by stirring in 75 g of 86% glycerin. 20 g vitamin E-acetate is added and distributed by continuous stirring. Homogenisation of this roughly dispersed inhomogeneous mixture by means of a high pressure homogenizer. A transparent emulsion-like solution with high viscosity results.

### EXAMPLE 2

5 g of a fraction of phospholipid from soya with a PC-content of 70% is dispersed by stirring in 75 g of a 70% fructosesolution. After the addition of 20 g of a medium chain triglyceride followed by high pressure homogenisation of this mixture a transparent emulsion-like solution with honey type viscosity will be obtained.

1. Emulsion-like water-soluble concentrates of fatlike substances by means of combinations of phopholipids or lecithins and highly concentrated aqueous solutions of polyols or carbohydrates with the concentration of the solutions of polyols or carbohydrates being from 30 mass-% to 99 mass-%, the ratio of lecithin: lipid being from 1:1 and 1:12 and the concentration of the mixture lecithin/lipid in solution of polyols or carbohydrates being from 10 mass-% to 90 mass-%.

2. Emulsion-like water-soluble concentrates according to claim 1, characterized in that the applied phopholipids or lecithins are either unsaturated, hydrogenated or hydroxylated, with the lecithins or phospholipids being extracted from soya, rape, fish, milk, or egg and the fractions of lecithin being formed of oil free fractions with parts of phosphatidylcholine from 10 mass-% to 100 mass-%.

3. Emulsion-like water-soluble concentrates according to claim 1, characterized in that lecithins contain at least 50 mass-% of substances not soluble in aceton, overwhelmingly from the group of the polar lipids, e. g. glycerinphosphatides, sphingophosphatides, spingoglycolipids, glyceringlycolipids or aminolipids.

4. Emulsion-like water-soluble concentrates according to claim 1, characterized in that multi-hydric alcohols are preferably used as polyols with chain lengths of C₃-C₆, as for example glycerin, treitols, pentitols or hexitols.

5. Emulsion-like water-soluble concentrates according to claim 1, characterized in that monosaccharides, disaccharides, maltitol and maltodextrins are used as carbohydrates.

6. Emulsion-like water-soluble concentrates according to claim 1, characterized in that no monohydric highly volatile alcohols (VOC) and/or ethoxylated or other synthetic tensids are used.

7. Emulsion-like water-soluble concentrates according to claim 1, characterized in that no preservatives are used.

8. Emulsion-like water-soluble concentrates according to claim 1, characterized in that for production high pressure homogenizers, ultrasound or rotor-stator-mixers are used.

9. Emulsion-like water-soluble concentrates according to claim 1, characterized in that preferably substances are used from the group of the lipids (e.g. fatty acids, waxes, wax esters, paraffins, fatty alcohols, fatty aldehydes, glycerides, etc.), Isoprenoides (terpenes and steroids: e.g. vitamin-A, vitamin-E, vitamin-D, vitamin-K, bisabolol, menthol, glucocorticoids, essential oils, cholesterol, sitosterols, coenzym Q 10, etc.), of the polar lipids (ceramides, sphingolipids, glycolipids, etc.) as well of the oil soluble UV-A and UV-B filter and silicon oils.

10. Application of the emulsion-like water-soluble concentrates according to at least one of the precedent claims for cosmetic, pharmaceutical or dietetic applications.

11. Application according to claim 9 characterized by topical, oral or parenteral application.
